(19) United States
(12) Patent Application Publication
(10) Pub. No.: US 2014/0272940 A1
(43) Pub. Date: Sep. 18, 2014

(54) METHODS FOR DETECTION OF MULTIPLE TARGET NUCLEIC ACIDS

(71) Applicant: LIFE TECHNOLOGIES CORPORATION, Carlsbad, CA (US)

(72) Inventors: Wayne Ge, Austin, TX (US); Craig Cummings, Pacifica, CA (US); Darcy Myers, Austin, TX (US); Quoc Hoang, Austin, TX (US); Catherine O'Connell, Austin, TX (US); Maxim Brevnov, Union City, CA (US); Daniel Kephart, Austin, TX (US)

(73) Assignee: LIFE TECHNOLOGIES CORPORATION, Carlsbad, CA (US)

(21) Appl. No.: 14/206,716

(22) Filed: Mar. 12, 2014

Related U.S. Application Data
(60) Provisional application No. 61/779,174, filed on Mar. 13, 2013.

(51) Int. Cl. C12Q 1/68

(52) U.S. Cl. CPC C12Q 1/6888
USPC 435/5; 435/6.12

(57) ABSTRACT

The present disclosure, in some embodiments, relates to compositions and methods for detection of multiple target nucleic acid sequences by a single nucleic acid amplification based assay (e.g. PCR). Compositions comprising two or more primers are described for multiple target nucleic acid detection. Methods for differential detection of microorganisms (including strains/serovars/subtypes thereof) and cell types comprise a single step method to detect a signature of target nucleic acid sequences comprising: two, three or more target nucleic acids that are uniquely present in a microorganism/strain/serovar/subtype and/or cell and absent in other closely related organisms/cells. Embodiments relate to methods of diagnosis of diseases or conditions that can be detected by detecting the presence of two or more nucleic acid target markers.

### CROSS-REFERENCE(S) TO RELATED APPLICATION(S)

This application claims priority under 35 U.S.C.§119(e) of U.S. Provisional Application No. 61/779,174, filed Mar. 13, 2013, the entire contents of which are incorporated herein by reference.

### FIELD

The present disclosure, in some embodiments, relates to compositions, kits and methods for detection of multiple target nucleic acids by a single nucleic acid amplification based assay (e.g. PCR). Some embodiments describe compositions and methods for detection and differential detection of microorganisms (bacteria, viruses, fungi) including strains/serovars/subtypes thereof and/or of a cell type by a single step method comprising the detection of two or more target nucleic acids that are uniquely present in a microorganism/strain/serovar/subtype or cell and absent in other closely related organisms/cell types. Some embodiments describe methods of diagnosis of diseases or conditions that can be detected by detecting the presence of two or more nucleic acid target markers (e.g. mutated genes or fragments, target nucleic acids of genes known to cause or be associated with a disease or condition, genes of microbes/viruses causing a disease, cancer markers, other nucleic acid sequences or regions that are characteristically present in a diseased cell).

### BACKGROUND

Microorganisms of various kinds, due to their constant and rapid evolution, can each have thousands of subtypes. However only a handful of subtypes of some microorganisms are responsible for the vast majority of human and animal diseases. These subtypes are the primary detection targets of detection and diagnostic methods due to their hazardous effects health.

One example is contamination of food and beverages by microbes that cause foodborne illnesses. The genotyping of microorganisms is important for food safety and environmental surveillance tasks. The bacterium Salmonella is the leading cause of food borne illness in the United States with an increasing number of reported Salmonella outbreaks originating from produce in recent years. Over 2,500 serovars of Salmonella have been identified to date. Ingestion of some Salmonella serovars can result in salmonellosis. Salmonellosis is characterized by diarrhea, fever, and abdominal cramps that typically arise 12 to 72 hours after infection. Salmonellosis usually lasts 4 to 7 days, and most people recover without treatment. However, in some cases Salmonella infection can enter the bloodstream leading to sepsis and can ultimately cause death if not treated. Children under the age of 5 years are most likely to get salmonellosis. Children, the elderly, and immunocompromised are most likely to have severe infections.

One of the pathogenic Salmonella subtypes that causes salmonellosis is Salmonella Hadar (S. Hadar). According to data from the CDC, 12 cases of S. Hadar linked to turkey burgers reported in 10 states of the U.S. of America between 12/27/10-4/1/11. To distinguish S. Hadar from all other subtypes of Salmonella, three gene targets need to be simultaneously detected. While real-time PCR is an important rapid molecular detection tool that has great potential to minimize the risk of Salmonella outbreaks it is challenging to develop and run a triplex real-time PCR assay to detect three target genes of S. Hadar simultaneously.

Another common food pathogens are STEC E. coli strains such as E. coli O157:H7. Differential detection of this pathogenic strain over other closely related E. coli strains (such as the 055:H7 strain) require the detection of two target nucleic acid sequences that are uniquely present in E. coli O157:H7.

Several other food borne pathogens, many viruses and other disease causing pathogens have a similar need for detection of two or more target nucleic acid sequences the combination of which are unique to that pathogen to differentially distinguish them over other non-pathogenic forms.

Similarly several diseases, such as some cancers, have multiple nucleic acid markers that are present in a diseased cell that are not present in a cell that is disease free. Current molecular methods are unable to detect the presence of multiple target nucleic acids in one assay. Performing two or more separate assays is time consuming and expensive. There exists a need for novel assays and methods for detecting multiple target nucleic acids by simple one step methods.

### SUMMARY

The present disclosure, in some embodiments, describes compositions and methods for detection of multiple target nucleic acids by a single step nucleic acid amplification based method.

Some embodiments describe compositions comprising a set of PCR primers for detecting multiple target nucleic acid sequences comprising: a first PCR primer comprising a reverse primer of a first target nucleic acid sequence; a second PCR primer comprising a reverse primer of a second target nucleic acid sequence; and a third PCR primer comprising either the 5′ terminus of a forward primer of the first target nucleic acid sequence or the 5′ terminus of a forward primer of a second target nucleic acid sequence and a tag sequence attached thereto, the tag sequence comprising either a reverse complement of the forward primer of the second target nucleic acid sequence or comprising a reverse complement of the forward primer of the first target nucleic acid sequence. A set of PCR primers as described here can detect at least two targets nucleic acid sequences if present on a nucleic acid to be tested (e.g., a sample derived nucleic acid).

In one example embodiment, a set of primers operable to detect at least two target nucleic acid sequences if present on a nucleic acid to be tested can comprise: a first PCR primer comprising a reverse primer of a first target nucleic acid sequence; a second PCR primer comprising a reverse primer of a second target nucleic acid sequence; and a third PCR primer comprising the 5′ terminus of a forward primer of the first target nucleic acid sequence and the tag sequence attached thereto comprising a reverse complement of the forward primer of the second target nucleic acid sequence.

In another example embodiment, a set of primers operable to detect at least two target nucleic acid sequences if present on a nucleic acid to be tested can comprise: a first PCR primer comprising a reverse primer of a first target nucleic acid sequence; a second PCR primer comprising a reverse primer of a second target nucleic acid sequence; and a third PCR primer comprising the 5′ terminus of a forward primer of a second target nucleic acid sequence and the tag sequence comprising a reverse complement of the forward primer of the first target nucleic acid sequence.

A PCR primer composition of the disclosure in some embodiments is described as comprising: a first PCR primer having a first contiguous nucleic acid sequence that can specifically hybridize to the 5′ terminus of a first target nucleic acid sequence joined to a second contiguous nucleic acid sequence that is a reverse complement of at least a part of the 5′ terminus of a second target nucleic acid sequence OR a first PCR primer having a first contiguous nucleic acid sequence that can specifically hybridize to the 5′ terminus of a first target nucleic acid sequence joined to a second contiguous nucleic acid sequence that is a reverse complement of at least a part of the 5′ terminus of a first target nucleic acid sequence; a second PCR primer that can specifically hybridize to the 3′ end of the first target nucleic acid sequence; a third PCR primer that is a reverse complement of at least a part of the 3′ terminus of the second target nucleic acid sequence.

Some embodiments describe compositions comprising a set of PCR primers for detecting multiple target nucleic acid sequences comprising: a first PCR primer comprising a reverse primer of a first target nucleic acid sequence; a second PCR primer comprising a reverse primer of a second target nucleic acid sequence; a third PCR primer comprising the 5′ terminus of a forward primer of the first target nucleic acid sequence joined to a tag sequence comprising a reverse complement of the forward primer of a third target nucleic acid sequence or the third PCR primer comprising the 5′ terminus of a forward primer of the second target nucleic acid sequence joined to a tag sequence of the third PCR primer comprising a reverse complement of the forward primer of a third target nucleic acid sequence; and a fourth PCR primer comprising the 5′ terminus of a forward primer of the second target nucleic acid sequence joined to a tag sequence comprising a reverse complement of the reverse primer of the third target nucleic acid sequence or a fourth PCR primer comprising the 5′ terminus of a forward primer of the first target nucleic acid sequence joined to a tag sequence of the fourth PCR primer comprising a reverse complement of the forward primer of a third target nucleic acid sequence.

A set of PCR primers as described here can detect at least three target nucleic acid sequences if present on a nucleic acid to be tested (e.g., a sample derived nucleic acid).

In one example embodiment, a set of primers operable to detect at least three targets nucleic acid sequences if present on a nucleic acid to be tested can comprise: a first PCR primer comprising a reverse primer of a first target nucleic acid sequence; a second PCR primer comprising a reverse primer of a second target nucleic acid sequence; a third PCR primer comprising the 5′ terminus of a forward primer of the first target nucleic acid sequence joined to a tag sequence comprising a reverse complement of the forward primer of a third target nucleic acid sequence; and a fourth PCR primer comprising the 5′ terminus of a forward primer of the second target nucleic acid sequence joined to a tag sequence comprising a reverse complement of the reverse primer of the third target nucleic acid sequence.

In another example embodiment, a set of primers operable to detect at least three target nucleic acid sequences if present on a nucleic acid to be tested can comprise: a first PCR primer comprising a reverse primer of a first target nucleic acid sequence; a second PCR primer comprising a reverse primer of a second target nucleic acid sequence; a third PCR primer comprising the 5′ terminus of a forward primer of the second target nucleic acid sequence joined to a tag sequence of the third PCR primer comprising a reverse complement of the forward primer of a third target nucleic acid sequence; and a fourth PCR primer comprising the 5′ terminus of a forward primer of the first target nucleic acid sequence joined to a tag sequence of the fourth PCR primer comprising a reverse complement of the forward primer of a third target nucleic acid sequence.

In some embodiments, PCR primer compositions of the disclosure can also be described as a first PCR primer having a first contiguous nucleic acid sequence that can specifically hybridize to the 5′ terminus of a first target nucleic acid sequence joined to a second contiguous nucleic acid sequence that is a reverse complement of at least a part of the 5′ terminus of a third target nucleic acid sequence; a second PCR primer that can specifically hybridize to the 3′ end of the first target nucleic acid sequence; a third PCR primer having a first contiguous nucleic acid sequence that can specifically hybridize to the 5′ terminus of the second target nucleic acid sequence joined to a second contiguous nucleic acid sequence that is a reverse complement of at least a part of the 3′ terminus of a third target nucleic acid sequence; a fourth PCR primer that can specifically hybridize to the 3′ end of the second target nucleic acid sequence.

In some embodiments, the present disclosure describes methods of detection of multiple nucleic acid sequences (multiple targets) in a sample nucleic acid comprising a single step nucleic acid amplification methods such as a polymerase chain reaction (PCR) based methods. Such a method involves providing a plurality of primer compositions according the present disclosure specific to hybridize to at least two or more target nucleic acid sequences to be detected (in various embodiments, compositions comprising: at least two primers, or at least three primers, at least four primers, or more) and other components and conditions for a PCR reaction and generating one or more additional primers to prime one or more additional target nucleic acid sequences desired to be detected. Accordingly, some embodiments describe methods of generating a new PCR primer.

A method for generating a new PCR primer set, according to some embodiments, comprises: contacting a nucleic acid having three target nucleic acid sequences (e.g., a nucleic acid in a sample) with a plurality of primers comprising: a first PCR primer having a first contiguous nucleic acid sequence that can specifically hybridize to the 5′ terminus of a first target nucleic acid sequence joined to a second contiguous nucleic acid sequence that is a reverse complement of at least a part of the 5′ terminus of a third target nucleic acid sequence; a second PCR primer that can specifically hybridize to the 3′ end of the first target nucleic acid sequence; a third PCR primer having a first contiguous nucleic acid sequence that can specifically hybridize to the 5′ terminus of the second target nucleic acid sequence joined to a second contiguous nucleic acid sequence that is a reverse complement of at least a part of the 3′ terminus of a third target nucleic acid sequence; and a fourth PCR primer that can specifically hybridize to the 3′ end of the second target nucleic acid sequence; providing conditions for amplification of the first target nucleic acid sequence primed by the first and the second PCR primers to amplify and generate a first amplicon comprising the first target nucleic acid sequence and at least a part of the 5′ terminus of the third target nucleic acid sequence; and co-amplifying the second target nucleic acid sequence primed by the third and fourth PCR primers to amplify and generate a second amplicon comprising the second target nucleic acid sequence and at least a part of the 3′ end of the third target nucleic acid sequence, wherein the first and second amplicons generated comprise the new PCR primers (new PCR primer set) that can specifically hybridize to and prime the amplification of the 3′ and the 5′ ends of the third nucleic acid sequence in the same PCR reaction. Accordingly a method of the disclosure can generate in vivo a new primer set in initial PCR cycles (by using primer compositions of the disclosure). The new primer set generated then in later PCR cycles hybridizes to and amplifies a third target nucleic acid sequence to be detected. In some embodiments, detection of three target nucleic acids in a sample will comprise detecting one or more amplicons generated during the PCR reaction.

A method for generating a new PCR primer, set according to some embodiments, comprises: contacting a nucleic acid having two target nucleic acid sequences (e.g., a nucleic acid in a sample) with a plurality of primers comprising: a first PCR primer having a first contiguous nucleic acid sequence that can specifically hybridize to the 5′ terminus of a first target nucleic acid sequence joined to a second contiguous nucleic acid sequence that is a reverse complement of at least a part of the 5′ terminus of a second target nucleic acid sequence; a second PCR primer that can specifically hybridize to the 3′ end of the first target nucleic acid sequence; and a third PCR primer that is a reverse complement of at least a part of the 3′ terminus of the second target nucleic acid sequence; and providing conditions for amplification of the first target nucleic acid sequence primed by the first and the second PCR primers to amplify and generate an amplicon comprising the first target nucleic acid sequence and at least a part of the 5′ terminus of the second target nucleic acid sequence; wherein the amplicons generated comprises the new PCR primer that can specifically hybridize to and prime the amplification of the 5′ ends of the second target nucleic acid sequence and can be used with the third primer to amplify the second target nucleic acid. In some embodiments, a method of the disclosure as described here can generate in vivo a new primer in initial PCR cycles that in later PCR cycles can hybridizes to and amplify a second target nucleic acid sequence to be detected. In some embodiments, detection of two target nucleic acid sequences in a sample will comprise detecting one or more amplicons generated during the PCR reaction.

Some embodiments describe methods for detecting multiple target nucleic acids in a sample comprising: contacting sample derived nucleic acids with at least three PCR primers comprising: a first PCR primer having a first contiguous nucleic acid sequence that can specifically hybridize to the 5′ terminus of a first target nucleic acid sequence joined to a second contiguous nucleic acid sequence that is a reverse complement of at least a part of the 5′ terminus of a second target nucleic acid sequence; a second PCR primer that can specifically hybridize to the 3′ end of the first target nucleic acid sequence; and a third PCR primer that is a reverse complement of at least a part of the 3′ terminus of the second target nucleic acid sequence; amplifying the first target nucleic acid sequence primed by the first and the second PCR primers to generate an amplicon comprising the first target nucleic acid sequence and at least a part of the 5′ terminus of the second target nucleic acid sequence; amplifying the second target nucleic acid using the amplicon generated as a fourth primer with the third primer to hybridize to and prime the amplification of the second target nucleic acid sequence to generate a second amplicon comprising a fusion of the second target nucleic acid sequence and part of the first target nucleic acid sequence; and detecting the presence of the second amplicon to detect the presence of the first and the second nucleic acid sequences in the sample derived nucleic acids. In some embodiments, detection of two target nucleic acid sequences in a sample will comprise detecting one or more amplicons generated during the PCR reaction.

In some embodiments of the method, the first and the second target nucleic acid sequences comprise a signature of target nucleic acid sequences unique to a cell type such as a microorganism, a bacteria, a virus, a fungi, a pathogen, a subtype of a microorganism, a serotype of a microorganism or virus, a strain of a virus or microorganism, a diseased cell, a cancerous cell, a stem cell and can be used to specifically detect the unique cell type

In some embodiments, methods for detecting multiple target nucleic acid sequences in a sample comprise: contacting a sample derived nucleic acid with at least four primers comprising: a first PCR primer having a first contiguous nucleic acid sequence that can specifically hybridize to the 5′ terminus of a first target nucleic acid sequence joined to a second contiguous nucleic acid sequence that is a reverse complement of at least a part of the 5′ terminus of a third target nucleic acid sequence; a second PCR primer that can specifically hybridize to the 3′ end of the first target nucleic acid sequence; a third PCR primer having a first contiguous nucleic acid sequence that can specifically hybridize to the 5′ terminus of the second target nucleic acid sequence joined to a second contiguous nucleic acid sequence that is a reverse complement of at least a part of the 3′ terminus of a third target nucleic acid sequence; and a fourth PCR primer that can specifically hybridize to the 3′ end of the second target nucleic acid sequence; amplifying the first target nucleic acid sequence primed by the first and the second PCR primers to amplify and generate a first amplicon comprising the first target nucleic acid sequence and at least a part of the 5′ terminus of the third target nucleic acid sequence; co-amplifying the second target nucleic acid sequence primed by the third and fourth PCR primers to amplify and generate a second amplicon comprising the second target nucleic acid sequence and at least a part of the 3′ end of the third target nucleic acid sequence, wherein the first and second amplicons generated comprise a new PCR primer set that can specifically hybridize to and prime the amplification of the third nucleic acid sequence; amplifying the third target nucleic acid using the first and second amplicons generated to generate a third amplicon comprising a fusion of the first, second and third target nucleic acid sequences; and detecting the presence of the third amplicon to detect the presence of the first, second and third nucleic acid sequences in the sample derived nucleic acids. In some embodiments, the first, second and the third target nucleic acid sequences comprise a signature of target nucleic acid sequences unique to a cell type such as a microorganism, a bacteria, a virus, a fungi, a pathogen, a subtype of a microorganism, a serotype of a microorganism or virus, a strain of a virus or microorganism, a diseased cell, a cancerous cell, a stem cell and can be used to specifically detect the unique cell type.

Some embodiment methods relate to detection and differential detection of microorganisms (bacteria, viruses, fungi) and strains/serovars/subtypes thereof by a single step amplification method comprising the detection of two or more target nucleic acids that are uniquely present in one microorganism/strain/serovar/subtypes and not in other closely related organisms. Some non-limiting examples include methods for detection of a strains and/or a subtype and/or a serovar of a Salmonella enterica subsp. enterica, or an E. coli, or a virus such as an influenza virus.

Some embodiments relate to detection and/or differential detection and/or diagnosis of diseases or conditions that can be detected by detecting the presence of two or more nucleic acid target markers (e.g. mutated genes or fragments, target nucleic acids of genes known to cause or be associated with a disease or condition, genes of microbes/viruses causing a disease, cancer markers, other nucleic acid sequences or regions that are characteristically present in a diseased cell).

A method of the disclosure to co-detect the presence of two or more target nucleic acids by a single assay, in some embodiments, further comprises using a single detectable label for detection. For example, in the methods outlined above the amplicon comprising the fusion of target nucleic acid sequences, i.e., either a second amplicon comprising a fusion of the second target nucleic acid sequence and part of the first target nucleic acid sequence; or a third amplicon comprising a fusion of the first, second and third target nucleic acid sequences, can be detected by a single label. In some non-limiting examples, the single label can be a DNA intercalating dye such as ethedium bromide, or SYBR green or a TaqMan® dye.

In some embodiments, detection of amplification products can be done by hybridization of a labeled probe with specificity to an amplicon following an amplification reaction to detect the generation of an amplicon.

In some embodiments, a nucleic acid amplification can be preceded or simultaneously coupled with hybridization of a labeled probe, such as a TaqMan® probe, to a target nucleic acid prior to the commencement of the polymerase reaction.

In some embodiments, not detecting any amplified product using methods described above can be used to exclude the presence of a microorganism of a particular serovar, subtype or strain; and/or exclude the presence of a cell of a certain type (cell expressing the multiple target nucleic acid sequences tested for). In some embodiments, detecting and/or identifying an amplified nucleic acid comprises one or more methods such as but not limited to hybridization, mass spectrometry, nanostring, microfluidics, chemiluminescence, enzyme technologies and combinations thereof. Some embodiments comprise identifying the particular target nucleic acid sequences in an amplicon can comprise methods such as DNA sequencing.

Some embodiments describe kits for detection of multiple target nucleic acid sequences in a sample. One embodiment kit can comprising: a first PCR primer having a first contiguous nucleic acid sequence that can specifically hybridize to the 5′ terminus of a first target nucleic acid sequence joined to a second contiguous nucleic acid sequence that is a reverse complement of at least a part of the 5′ terminus of a second target nucleic acid sequence; a second PCR primer that can specifically hybridize to the 3′ end of the first target nucleic acid sequence; a third PCR primer that is a reverse complement of at least a part of the 3′ terminus of the second target nucleic acid sequence; and PCR reagents, buffers and an instruction protocol.

Another kit embodiment can comprise: a first PCR primer having a first contiguous nucleic acid sequence that can specifically hybridize to the 5′ terminus of a first target nucleic acid sequence joined to a second contiguous nucleic acid sequence (or tag sequence) that is a reverse complement of at least a part of the 5′ terminus of a third target nucleic acid sequence; a second PCR primer that can specifically hybridize to the 3′ end of the first target nucleic acid sequence; a third PCR primer having a first contiguous nucleic acid sequence that can specifically hybridize to the 5′ terminus of the second target nucleic acid sequence joined to a second contiguous nucleic acid sequence (or a tag sequence) that is a reverse complement of at least a part of the 3′ terminus of a third target nucleic acid sequence; a fourth PCR primer that can specifically hybridize to the 3′ end of the second target nucleic acid sequence; and PCR reagents, buffers and an instruction protocol.

A wide variety of samples can be tested by the present methods using one or more kits and/or compositions as described herein. Microorganisms and pathogens are often present as contaminants in a sample. Samples that can be tested by methods of the disclosure to detect a microbial contaminant therein include but are not limited to a food sample (processed food samples and raw food samples), a beverage sample, an agricultural sample, a produce sample, an animal sample, a clinical sample, a veterinary sample, an environmental sample, a biological sample, a water sample and an air sample.

In some embodiment methods described here, nucleic acids (RNA/DNA) can be extracted from a sample suspected to contain a microorganism to be detected prior to amplification or detection. A method can further comprise one or more steps such as: enrichment of microorganisms in a sample prior to nucleic acid extraction; and/or nucleic acid extraction from microorganisms in a sample; and/or nucleic acid isolation from a sample; and/or nucleic acid purification from a sample; and/or lysing bacterial cells from a sample prior to hybridization/amplification with primers and/or probes.

In some embodiment methods described here, nucleic acids (RNA/DNA) can be extracted from a cell/tissue suspected to contain the multiple nucleic acid target sequences to be detected prior to amplification or detection.

In some embodiments, methods of the disclosure can be performed on an automated system. Automation decreases the time as well as efficiency and allows processing multiples samples. Automated systems may comprise platforms to automate sample preparation such as but not limited to MagMAX™ Express-96 Magnetic Particle Processor by Life Technologies Corporation; Pathatrix system by Life Technologies (http://www.matrixmsci.com/pathatrix.htm); MagNA Pure System by Roche; the QIAsymphony system by Qiagen, among others.

Methods can comprise a nucleic acid amplification reaction including PCR, end-point determination, quantitative real-time PCR methods such as a SYBR® Green Assay or a TaqMan® Assay. Methods can comprise hybridization using probes specific to amplified nucleic acid sequences encoding one or more target sequences. Combinations of amplification and hybridization may be used for detection according to some embodiments.

In some embodiments, hybridization may comprise at least a first probe and a second probe, the first probe further comprising a first label and said second probe further comprising a second label, wherein both labels are selected from a dye, a radioactive isotope, a chemiluminescent label, and an enzyme, the dye comprises a fluorescein dye, a rhodamine dye, or a cyanine dye, the dye is a fluorescein dye and first probe is labeled with FAM™ dye and said second probe is labeled with VIC® dye.

In the following detailed description, certain aspects and embodiments will become evident. It should be understood that a given embodiment need not have all aspects and features described herein. It should be understood that these aspects and embodiments are merely exemplary and explanatory and are not restrictive of the present disclosure. It is to be understood that both the foregoing general description and the following detailed description are exemplary and explanatory only and are not restrictive of the present disclosure, as claimed.

### BRIEF DESCRIPTION OF THE DRAWINGS

FIG. 1 is a schematic view showing a design of one example embodiment method of the present disclosure showing a PCR reaction with several primers to amplify and detect the co-existence of three target nucleic acid sequences depicted as Target 1, 2 and 3;

FIG. 2 is an agarose gel electrophoresis result showing the production of fused amplicons of three targets that collectively identify Salmonella Hadar strain according to one example embodiment method of the present disclosure;

FIGS. 3A and 3B are real-time quantitative results of an example method of the present disclosure showing in FIG. 3A. high sensitivity of the assay; and in FIG. 3B. showing linearity with initial input Salmonella Hadar DNA copy numbers;

FIG. 4 is a schematic view showing a design of one example embodiment method of the present disclosure showing a PCR primer design to amplify and detect the co-existence of two target genes shown as target 1 and 2; and

FIG. 5 is an agarose gel electrophoresis result of an example method of the present disclosure showing the production of fused amplicons of two targets that collectively identify O157:H7 E. coli strain.

### DETAILED DESCRIPTION

For the purposes of interpreting of this specification, the following definitions may apply and whenever appropriate, terms used in the singular will also include the plural and vice versa. In the event that any definition set forth below conflicts with the usage of that word in any other document, including any document incorporated herein by reference, the definition set forth below shall always control for purposes of interpreting this specification and its associated claims unless a contrary meaning is clearly intended (for example in the document where the term is originally used). It is noted that, as used in this specification and the appended claims, the singular forms “a,” “an,” and “the,” include plural referents unless expressly and unequivocally limited to one referent. The use of “or” means “and/or” unless stated otherwise. The use of “comprise,” “comprises,” “comprising,” “include,” “includes,” and “including” are interchangeable and not intended to be limiting. Furthermore, where the description of one or more embodiments uses the term “comprising,” those skilled in the art would understand that, in some specific instances, the embodiment or embodiments can be alternatively described using the language “consisting essentially of” and/or “consisting of:”

As used herein, the phrase “nucleic acid,” “nucleic acid sequence,” “oligonucleotide”, and “polynucleotides” are interchangeable and not intended to be limiting. As used herein, the term “polynucleotide” refers to a polymeric form of nucleotides of any length, either ribonucleotides, deoxynucleotides, or peptide nucleic acids (PNA), and includes both double- and single-stranded RNA, DNA, and PNA. A polynucleotide may include nucleotide sequences having different functions, including, for instance, coding regions, and non-coding regions such as regulatory regions. A polynucleotide can be obtained directly from a natural source, or can be prepared with the aid of recombinant, enzymatic, or chemical techniques. A polynucleotide can be linear or circular in topology. A polynucleotide can be, for example, a portion of a vector, such as an expression or cloning vector, or a fragment. An “oligonucleotide” refers to a polynucleotide of the present disclosure, typically a primer and/or a probe.

As used herein, the phrase “hybridization conditions” or “stringent hybridization conditions” refers to hybridization conditions which can take place under a number of pH, salt and temperature conditions. The pH can vary from 6 to 9, preferably 6.8 to 8.5. The salt concentration can vary from 0.15 M sodium to 0.9 M sodium, and other cations can be used as long as the ionic strength is equivalent to that specified for sodium. The temperature of the hybridization reaction can vary from 30° C. to 80° C., preferably from 45° C. to 70° C. Additionally, other compounds can be added to a hybridization reaction to promote specific hybridization at lower temperatures, such as at or approaching room temperature. Among the compounds contemplated for lowering the temperature requirements is formamide. Thus, a polynucleotide is typically “substantially complementary” to a second polynucleotide if hybridization occurs between the polynucleotide and the second polynucleotide. As used herein, “hybridization” or “specific hybridization” refers to hybridization between two polynucleotides under stringent hybridization conditions.

As used herein a “target-specific polynucleotide” refers to a polynucleotide having a target-binding segment that is perfectly or substantially complementary to a target sequence, such that the polynucleotide binds specifically to an intended target without significant binding to non-target sequences under sufficiently stringent hybridization conditions. A target-specific polynucleotide can be e.g., a primer or probe and can hybridize with its complementary target sequence by hybridization.

The terms “target sequence”, “target nucleic acid region,” “target specific nucleic acid,” “target region,” “target signature sequence” “target nucleic acid(s)”, “target nucleic acid sequences,” “target” or “target polynucleotide sequence” refers to a nucleic acid of interest. Example targets of interest in some embodiments of this application include nucleic acid regions that are unique (uniquely present in) or specific (specifically present in) to one or more microorganisms including bacteria, viruses, fungi. In some embodiments, subtypes and/or strains and/or serovars of microorganisms can comprise a signature of target sequences. For example, the presence of “target nucleic acid a,” “target nucleic acid b” and “target nucleic acid c” may be unique to “microorganism 1” and hence the signature of target sequences unique to “microorganism 1;” while the presence of “target nucleic acid a,” “target nucleic acid b” and “target nucleic acid d” may comprise the unique signature target sequences of “microorganism 2.”

Other example target nucleic acids of interest, in some embodiments of this application, include signature target sequences of a particular cell type (present in a cell type and not present in another cell type).

In some embodiments, “target nucleic acid sequences” can comprise fragments of a larger target sequences, complements as well as minor mutations including sequences with about 90% homology to a target nucleic acid sequence.

A target sequence can be a polynucleotide sequence that is the subject of hybridization with a complementary polynucleotide, e.g. a primer or probe. The target sequence can be composed of DNA, RNA, an analog thereof, and including combinations thereof.

In molecular methods described herein the amplification of one or more target nucleic acid sequences is desired to detect the presence of one or more target nucleic acid sequences. Presence of one or more target nucleic acid sequences is indicative of the subtype and/or strain and/or serovar of a microorganism or type of cell. A target sequence may be known or not known, in terms of its actual sequence and/or function. A target sequence may or may not be of biological significance. Target sequences may include regions of genomic DNA, regions of genomic DNA which are believed to contain one or more polymorphic sites, DNA encoding or believed to encode genes or portions of genes of known or unknown function, DNA encoding or believed to encode proteins or portions of proteins of known or unknown function, DNA encoding or believed to encode regulatory regions such as promoter sequences, splicing signals, polyadenylation signals, etc.

It is expected that minor sequence variations in specific target nucleotide sequences associated with nucleotide additions, deletions and mutations, whether naturally occurring or introduced in vitro, would not interfere with the usefulness of the methods and primers and probes disclosed herein, as would be understood by one of skill in the art. Therefore, the scope of the present disclosure is intended to encompass minor variations in the sequences of described here and sequences having at least 90% homology to the primer and probe sequences disclosed herein.

As used herein an “amplified target sequence product” or “amplified product” or “amplicon” refers to the resulting amplicon from an amplification reaction such as a polymerase chain reaction. The resulting amplicon product arises from hybridization of complementary primers to a target polynucleotide sequence under suitable hybridization conditions and the repeating in a cyclic manner the polymerase chain reaction as catalyzed by DNA polymerase for DNA amplification or RNA polymerase for RNA amplification.

As used herein, the “polymerase chain reaction” or PCR is an amplification of nucleic acid consisting of an initial denaturation step which separates the strands of a double stranded nucleic acid sample, followed by repetition of (i) an annealing step, which allows amplification primers to anneal specifically to positions flanking a target sequence; (ii) an extension step which extends the primers in a 5′ to 3′ direction thereby forming an amplicon polynucleotide complementary to the target sequence, and (iii) a denaturation step which causes the separation of the amplicon from the target sequence (Mullis et al., eds, The Polymerase Chain Reaction, BirkHauser, Boston, Mass. (1994). Each of the above steps may be conducted at a different temperature, preferably using an automated thermocycler (Applied Biosystems LLC, a division of Life Technologies Corporation, Foster City, Calif.). If desired, such as if the presence of a target sequence on an RNA is desired to be known, RNA samples can be converted to DNA/RNA heteroduplexes or to duplex cDNA by methods such as reverse transcription which known to one of skill in the art.

As used herein, “amplifying” and “amplification” refers to a broad range of techniques for increasing polynucleotide sequences, either linearly or exponentially. Exemplary amplification techniques include, but are not limited to, PCR or any other method employing a primer extension step. Other non-limiting examples of amplification include, but are not limited to, ligase detection reaction (LDR) and ligase chain reaction (LCR). Amplification methods may comprise thermal-cycling or may be performed isothermally. In various embodiments, the term “amplification product” or “amplified product” includes products from any number of cycles of amplification reactions.

In certain embodiments, amplification methods comprise at least one cycle of amplification, for example, but not limited to, the sequential procedures of: hybridizing primers to primer-specific portions of target sequence or amplification products from any number of cycles of an amplification reaction; synthesizing a strand of nucleotides in a template-dependent manner using a polymerase; and denaturing the newly-formed nucleic acid duplex to separate the strands. The cycle may or may not be repeated.

Descriptions of certain amplification techniques can be found, among other places, in H. Ehrlich et al., Science, 252:1643-50 (1991), M. Innis et al., PCR Protocols: A Guide to Methods and Applications, Academic Press, New York, N.Y. (1990), R. Favis et al., Nature Biotechnology 18:561-64 (2000), and H. F. Rabenau et al., Infection 28:97-102 (2000); Sambrook and Russell, Molecular Cloning, Third Edition, Cold Spring Harbor Press (2000) (hereinafter “Sambrook and Russell”), Ausubel et al., Current Protocols in Molecular Biology (1993) including supplements through September 2005, John Wiley & Sons (hereinafter “Ausubel et al.”).

The term “label” refers to any moiety which can be attached to a molecule and (i) provides a detectable signal; (ii) interacts with a second label to modify the detectable signal provided by the second label, e.g. FRET; (iii) stabilizes hybridization, i.e. duplex formation; or (iv) provides a capture moiety, i.e. affinity, antibody/antigen, ionic complexation. Labeling can be accomplished using any one of a large number of known techniques employing known labels, linkages, linking groups, reagents, reaction conditions, and analysis and purification methods. Labels include light-emitting compounds which generate a detectable signal by fluorescence, chemiluminescence, or bioluminescence (Kricka, L. in Nonisotopic DNA Probe Techniques (1992), Academic Press, San Diego, pp. 3-28). Another class of labels are hybridization-stabilizing moieties which serve to enhance, stabilize, or influence hybridization of duplexes, e.g. intercalators, minor-groove binders, and cross-linking functional groups (Blackburn, G. and Gait, M. Eds. “DNA and RNA structure” in Nucleic Acids in Chemistry and Biology, 2.sup.nd Edition, (1996) Oxford University Press, pp. 15-81). Yet another class of labels effect the separation or immobilization of a molecule by specific or non-specific capture, for example biotin, digoxigenin, and other haptens (Andrus, A. “Chemical methods for 5′ non-isotopic labeling of PCR probes and primers” (1995) in PCR 2: A Practical Approach, Oxford University Press, Oxford, pp. 39-54).

The terms “annealing” and “hybridization” are used interchangeably and mean the base-pairing interaction of one nucleic acid with another nucleic acid that results in formation of a duplex or other higher-ordered structure. The primary interaction is base specific, i.e. A/T and G/C, by Watson/Crick and Hoogsteen-type hydrogen bonding.

The term “end-point analysis” refers to a method where data collection occurs only when a reaction is substantially complete.

The term “real-time analysis” refers to periodic monitoring during PCR. Certain systems such as the Applied Biosystems 7500 Real-Time PCR System (Applied Biosystems, Foster City, Calif.) conduct monitoring during each thermal cycle at a pre-determined or user-defined point. Real-time analysis of PCR with FRET probes measures fluorescent dye signal changes from cycle-to-cycle, preferably minus any internal control signals.

The term “quenching” refers to a decrease in fluorescence of a first moiety (reporter dye) caused by a second moiety (quencher) regardless of the mechanism.

A “primer,” as used herein, is an oligonucleotide that is complementary to a portion of target polynucleotide and, after hybridization to the target polynucleotide, may serve as a starting-point for an amplification reaction and the synthesis of an amplification product. Primers include, but are not limited to, spanning primers. A “primer pair” refers to two primers (forward and reverse primers) that can be used together for an amplification reaction of a target nucleic acid. One primer (forward primer) will hybridize to one end of a target nucleic acid sequence and another primer (reverse primer) will hybridize to the other end of the target nucleic acid, thereby priming the amplification of the entire target nucleic acid sequence. A “PCR primer” refers to a primer in a set of at least two primers that are capable of exponentially amplifying a target nucleic acid sequence in the polymerase chain reaction.

The term “probe” comprises a polynucleotide that comprises a specific portion designed to hybridize in a sequence-specific manner with a complementary region of a specific nucleic acid sequence, e.g., a target nucleic acid sequence. In certain embodiments, the specific portion of the probe may be specific for a particular sequence, or alternatively, may be degenerate, e.g., specific for a set of sequences. In certain embodiments, the probe is labeled. The probe can be an oligonucleotide that is complementary to at least a portion of an amplification product formed using two primers. A probe may be RNA or DNA. Depending on the detection means employed, the probe may be unlabeled, radiolabeled, chemiluminescent labeled, enzyme labeled, or labeled with a dye. The probe may be hybridized with a sample in solution or immobilized on a solid support such as nitrocellulose, a microarray or a nylon membrane, or the probe may be immobilized on a solid support, such as a silicon chip or a microarray.

The terms “complement” and “complementary” as used herein, refer to the ability of two single stranded polynucleotides (for instance, a primer and a target polynucleotide) to base pair with each other, where an adenine on one strand of a polynucleotide will base pair to a thymine or uracil on a strand of a second polynucleotide and a cytosine on one strand of a polynucleotide will base pair to a guanine on a strand of a second polynucleotide. Two polynucleotides are complementary to each other when a nucleotide sequence in one polynucleotide can base pair with a nucleotide sequence in a second polynucleotide. For instance, 5′-ATGC and 5′-GCAT are complementary.

A “label” refers to a moiety attached (covalently or non-covalently), or capable of being attached, to an oligonucleotide, which provides or is capable of providing information about the oligonucleotide (e.g., descriptive or identifying information about the oligonucleotide) or another polynucleotide with which the labeled oligonucleotide interacts (e.g., hybridizes). Labels can be used to provide a detectable (and optionally quantifiable) signal. Labels can also be used to attach an oligonucleotide to a surface.

A “fluorophore” is a moiety that can emit light of a particular wavelength following absorbance of light of shorter wavelength. The wavelength of the light emitted by a particular fluorophore is characteristic of that fluorophore. Thus, a particular fluorophore can be detected by detecting light of an appropriate wavelength following excitation of the fluorophore with light of shorter wavelength.

The term “quencher” as used herein refers to a moiety that absorbs energy emitted from a fluorophore, or otherwise interferes with the ability of the fluorescent dye to emit light. A quencher can re-emit the energy absorbed from a fluorophore in a signal characteristic for that quencher, and thus a quencher can also act as a fluorophore (a fluorescent quencher). This phenomenon is generally known as fluorescent resonance energy transfer (FRET). Alternatively, a quencher can dissipate the energy absorbed from a fluorophore as heat (a non-fluorescent quencher).

As used herein the term “sample” refers to a starting material suspected of harboring a particular microorganism or group of microorganisms. A “contaminated sample” refers to a sample harboring a pathogenic microrganism thereby comprising nucleic acid material from the pathogenic microbe. Examples of samples include, but are not limited to, food samples (including but not limited to samples from food intended for human or animal consumption such as processed foods, raw food material, produce (e.g., fruit and vegetables), legumes, meats (from livestock animals and/or game animals), fish, sea food, nuts, beverages, drinks, fermentation broths, and/or a selectively enriched food matrix comprising any of the above listed foods), water samples, environmental samples (e.g., soil samples, dirt samples, garbage samples, sewage samples, industrial effluent samples, air samples, or water samples from a variety of water bodies such as lakes, rivers, ponds etc.), air samples (from the environment or from a room or a building), clinical samples, samples obtained from humans suspected of having a disease or condition, veterinary samples, forensic samples, agricultural samples, pharmaceutical samples, biopharmaceutical samples, samples from food processing and manufacturing surfaces, and/or biological samples. In embodiments where cells are being tested for a disease or condition characterized by presence of multiple nucleic acid sequences as markers of the disease or condition or of infection by a pathogen/virus, a sample can comprise a cell, a tissue, a biopsy sample, a bodily fluid and the like.

Disclosed in some embodiments are methods and kits for the specific detection of microorganisms from samples including clinical samples, veterinary samples, food samples, complex food matrices, water, a beverage sample, a fermentation broth, a forensic sample, an environmental sample (e.g., soil, dirt, garbage, sewage, air, or water), including food processing and manufacturing surfaces, and/or biological samples.

A sample may be tested directly, or may be prepared or processed in some manner prior to testing. For example, a sample may be processed to enrich any contaminating microbe and may be further processed to separate and/or lyse microbial cells/viral cells/fungal cells contained therein. Lysed microbial cells from a sample may be additionally processed or prepares to separate, isolate and/or extract genetic material from the microbe for analysis to detect and/or identify the contaminating microbe. Some embodiments refer to “a nucleic acid in a sample” or a “sample derived nucleic acid” and refer to a nucleic acid comprised in a sample or obtained from a sample. Such nucleic acids can be tested by methods and using compositions described herein. In some embodiments described here, as sample may be subject to separation to initially separate microbes of interest from other microbes and other sample components. For example, for complex food samples with complex components separation methods can be used to separate microorganisms from food. Separated microbes from samples can also be enriched prior to analysis. Analysis of a sample may include one or more molecular methods. For example, according to some exemplary embodiments of the present disclosure, a sample may be subject to nucleic acid amplification (for example by PCR) using appropriate oligonucleotide primers that are specific to one or more microbe nucleic acid sequences that the sample is suspected of being contaminated with. Amplification products may then be further subject to testing with specific probes (or reporter probes) to allow detection of microbial nucleic acid sequences that have been amplified from the sample. In some embodiments, if a microbial nucleic acid sequence is amplified from a sample, further analysis may be performed on the amplification product to further identify, quantify and analyze the detected microbe (determine parameters such as but not limited to the microbial strain, pathogenecity, quantity etc.).

As used herein “preparing” or “preparing a sample” or “processing” or processing a sample” refers to one or more of the following steps to achieve separation of microbes from sample components and in some embodiments optionally extraction and/or separation of a nucleic acid from a sample: (1) optional separation of bacterial cells from sample components (such as a food sample), (2) optional bacterial enrichment, (3) optional cell lysis, and/or (4) optionally nucleic acid extraction and/or purification (e.g., DNA extraction, total nucleic acid extraction (i.e., DNA and RNA), genomic DNA extraction, RNA extraction). Types of nucleic acid extracted include, but are not limited to, DNA, RNA, mRNA and miRNA.

As used herein, “presence” refers to the existence (and therefore to the detection) of a reaction, a product of a method or a process (including but not limited to, an amplification product resulting from an amplification reaction), or to the “presence” and “detection” of an organism such as a pathogenic organism or a particular strain or species of an organism.

As used herein, “detecting” or “detection” refers to the disclosure or revelation of the presence or absence in a sample of a target polynucleotide sequence or amplified target polynucleotide sequence product. The detecting can be by end point, real-time, enzymatic, and by resolving the amplification product on a gel and determining whether the expected amplification product is present, or other methods known to one of skill in the art.

The presence or absence of an amplified product can be determined or its amount measured. Detecting an amplified product can be conducted by standard methods well known in the art and used routinely. The detecting may occur, for instance, after multiple amplification cycles have been run (typically referred to an end-point analysis), or during each amplification cycle (typically referred to as real-time). Detecting an amplification product after multiple amplification cycles have been run is easily accomplished by, for instance, resolving the amplification product on a gel and determining whether the expected amplification product is present. In order to facilitate real-time detection or quantification of the amplification products, one or more of the primers and/or probes used in the amplification reaction can be labeled, and various formats are available for generating a detectable signal that indicates an amplification product is present. For example, a convenient label is typically a label that is fluorescent, which may be used in various formats including, but are not limited to, the use of donor fluorophore labels, acceptor fluorophore labels, fluorophores, quenchers, and combinations thereof. Assays using these various formats may include the use of one or more primers that are labeled (for instance, scorpions primers, amplifluor primers), one or more probes that are labeled (for instance, adjacent probes, TaqMan® probes, light-up probes, molecular beacons), or a combination thereof. The skilled person in view of the present teachings will understand that in addition to these known formats, new types of formats are routinely disclosed. The present disclosure is not limited by the type of method or the types of probes and/or primers used to detect an amplified product. Using appropriate labels (for example, different fluorophores) it is possible to combine (multiplex) the results of several different primer pairs (and, optionally, probes if they are present) in a single reaction. As an alternative to detection using a labeled primer and/or probe, an amplification product can be detected using a polynucleotide binding dye such as a fluorescent DNA binding dye. Examples include, for instance, SYBR® Green dye or SYBR® Gold dye (Molecular Probes). Upon interaction with the double-stranded amplification product, such polynucleotide binding dyes emit a fluorescence signal after excitation with light at a suitable wavelength. A polynucleotide binding dye such as a polynucleotide intercalating dye also can be used.

Conditions that “allow” an event to occur or conditions that are “suitable” for an event to occur, such as hybridization, strand extension, and the like, or “suitable” conditions are conditions that do not prevent such events from occurring. Thus, these conditions permit, enhance, facilitate, and/or are conducive to the event. Such conditions, known in the art and described herein, may depend upon, for example, the nature of the nucleotide sequence, temperature, and buffer conditions. These conditions may also depend on what event is desired, such as hybridization, cleavage, or strand extension. An “extracted” polynucleotide refers to a polynucleotide that has been removed from a cell. An “isolated” polynucleotide refers to a polynucleotide that has been removed from its natural environment. A “purified” polynucleotide is one that is at least about 60% free, preferably at least about 75% free, and most preferably at least about 90% free from other components with which they are naturally associated.

The words “preferred” and “preferably” refer to embodiments of the present disclosure that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the present disclosure.

The terms “comprises” and variations thereof do not have a limiting meaning where these terms appear in the description and claims. Unless otherwise specified, “a,” “an,” “the,” and “at least one” are used interchangeably and mean one or more than one.

Also herein, the recitations of numerical ranges by endpoints include all numbers subsumed within that range (e.g., 1 to 5 includes 1, 1.5, 2, 2.75, 3, 3.80, 4, 5, etc.). The term “and/or” means one or all of the listed elements or a combination of any two or more of the listed elements.

There are many known methods of amplifying nucleic acid sequences including e.g., PCR. See, e.g., PCR Technology: Principles and Applications for DNA Amplification (ed. H. A. Erlich, Freeman Press, NY, N.Y., 1992); PCR Protocols: A Guide to Methods and Applications (eds. Innis, et al., Academic Press, San Diego, Calif., 1990); Mattila et al., Nucleic Acids Res. 19, 4967 (1991); Eckert et al., PCR Methods and Applications 1, 17 (1991); PCR (eds. McPherson et al., IRL Press, Oxford); and U.S. Pat. Nos. 4,683,202, 4,683,195, 4,800,159 4,965,188 and 5,333,675 each of which is incorporated herein by reference in their entireties for all purposes.

Nucleic acid amplification techniques are traditionally classified according to the temperature requirements of the amplification process. Isothermal amplifications are conducted at a constant temperature, in contrast to amplifications that require cycling between high and low temperatures. Examples of isothermal amplification techniques are: Strand Displacement Amplification (SDA; Walker et al., 1992, Proc. Natl. Acad. Sci. USA 89:392 396; Walker et al., 1992, Nuc. Acids. Res. 20:1691 1696; and EP 0 497 272, all of which are incorporated herein by reference), self-sustained sequence replication (3SR; Guatelli et al., 1990, Proc. Natl. Acad. Sci. USA 87:1874 1878), the Q.beta. replicase system (Lizardi et al., 1988, BioTechnology 6:1197 1202), and the techniques disclosed in WO 90/10064 and WO 91/03573.

Examples of techniques that require temperature cycling are: polymerase chain reaction (PCR; Saiki et al., 1985, Science 230:1350 1354), ligase chain reaction (LCR; Wu et al., 1989, Genomics 4:560 569; Barringer et al., 1990, Gene 89:117 122; Barany, 1991, Proc. Natl. Acad. Sci. USA 88:189 193), transcription-based amplification (Kwoh et al., 1989, Proc. Natl. Acad. Sci. USA 86:1173 1177) and restriction amplification (U.S. Pat. No. 5,102,784).

Other exemplary techniques include Nucleic Acid Sequence-Based Amplification (“NASBA”; see U.S. Pat. No. 5,130,238), Qβ replicase system (see Lizardi et al., BioTechnology 6:1197 (1988)), and Rolling Circle Amplification (see Lizardi et al., Nat Genet. 19:225 232 (1998)). The amplification primers of the present disclosure may be used to carry out, for example, but not limited to, PCR, SDA or tSDA. Any of the amplification techniques and methods disclosed herein can be used to practice the claimed present disclosure as would be understood by one of ordinary skill in the art.

PCR is an extremely powerful technique for amplifying specific polynucleotide sequences, including genomic DNA, single-stranded cDNA, and mRNA among others. Various methods of conducting PCR amplification and primer design and construction for PCR amplification will be known to those of skill in the art. Generally, in PCR a double-stranded DNA to be amplified is denatured by heating the sample. New DNA synthesis is then primed by hybridizing primers to the target sequence in the presence of DNA polymerase and excess dNTPs. In subsequent cycles, the primers hybridize to the newly synthesized DNA to produce discreet products with the primer sequences at either end. The products accumulate exponentially with each successive round of amplification.

The DNA polymerase used in PCR is often a thermostable polymerase. This allows the enzyme to continue functioning after repeated cycles of heating necessary to denature the double-stranded DNA. Polymerases that are useful for PCR include, for example, Taq DNA polymerase, Tth DNA polymerase, Tfl DNA polymerase, Tma DNA polymerase, Tli DNA polymerase, and Pfu DNA polymerase. There are many commercially available modified forms of these enzymes including: AmpliTaq® and AmpliTaq Gold® both available from Applied Biosystems. Many are available with or without a 3- to 5′ proofreading exonuclease activity. See, for example, Vent® and Vent®. (exo-) available from New England Biolabs.

Other suitable amplification methods include the ligase chain reaction (LCR) (e.g., Wu and Wallace, Genomics 4, 560 (1989) and Landegren et al., Science 241, 1077 (1988)), transcription amplification (Kwoh et al., Proc. Natl. Acad. Sci. USA 86, 1173 (1989)), and self-sustained sequence replication (Guatelli et al., Proc. Nat. Acad. Sci. USA, 87, 1874 (1990)) and nucleic acid based sequence amplification (NABSA). (See, U.S. Pat. Nos. 5,409,818, 5,554,517, and 6,063,603). The latter two amplification methods include isothermal reactions based on isothermal transcription, which produce both single-stranded RNA (ssRNA) and double-stranded DNA (dsDNA) as the amplification products in a ratio of about 30 or 100 to 1, respectively.

The present disclosure, in some embodiments, describes methods to detect the presence of multiple target nucleic acid sequences by a single nucleic acid amplification step method (e.g., single PCR step method). In some embodiments, detecting the presence of multiple target nucleic acid sequences detects and/or differentially detects the presence of a microorganism. For example, the detection of a pathogenic Salmonella subtype called Salmonella Hadar (S. Hadar) without cross-contamination of other subtypes of Salmonella requires the simultaneous detection of three gene targets (i.e., three target nucleic acid sequences). While this can be achieved by real-time PCR methods involving two or three separate PCR reactions it is challenging to develop and run a duplex and/or a triplex real-time PCR assay to detect the presence of three target genes of S. Hadar simultaneously. However, two or three separate PCR reactions are time consuming and not very economically conducive while testing large samples of food.

Several other food borne pathogens, viruses and other pathogens that cause diseases in animals and humans are similar in that there is a need for detection of a signature of target nucleic acid sequences, i.e., two or more target nucleic acid sequences have to be co-detected, the combinations of which are unique to that pathogen/virus to be able to differentially distinguish that pathogen/virus over other non-pathogenic forms. Similarly several diseases, such as some cancer types, have multiple nucleic acid markers that are present in a diseased cell that define the particular disease. As described above, current molecular methods such as real-time PCR are unable to detect the presence of multiple target nucleic acids in one assay. The present methods overcome these deficiencies in the art and provide one step nucleic acid amplification methods that are able to detect two or more target nucleic acids.

Although some specific examples are described in relation to detection of a S. Hadar microorganism and detection of an E. coli O157:H7 microorganism in food samples, one of skill in the art, in light of the present specification, will realize that the methods described herein can be performed on any sample type for the detection of any pathogen as well as in cells of any human, animal or plant where simultaneous multiple target nucleic acid detection is desired for any purpose. Accordingly the scope of this specification extends beyond the exemplary embodiments described and the described examples are merely for descriptive purpose.

[chemical structure]

FIG. 1, reproduced above for convenience, illustrates schematically an example method of the disclosure for amplifying multiple target nucleic acid sequences termed herein as “a hopping PCR method” and illustrates PCR primers designed herein to amplify and detect the co-existence of three target nucleic acid sequences shown as Target 1, Target 2 and Target 3. Four primers are shown in FIG. 1 comprising: 1) “a tag sequence” comprising the reverse complimentary sequence of forward primer of Target 3 (Rc-F3) is attached/joined to the 5′ terminus of forward primer of Target 1 (F1) and forms Intermediate Primer 1; 2) “another tag sequence” comprising the reverse complimentary sequence of reverse primer of Target 3 (Rc-R3) is attached/joined to the 5′ terminus of forward primer for Target 2 (F2) and form intermediate primer 2; 3) R1 the reverse primer of Target 1; and 4) R2 the reverse primer of Target 2.

During the initial PCR cycles, the reverse primer of Target 1, i.e, the R1 primer and Intermediate Primer 1 (Rc-F3-F1) comprising the reverse complimentary sequence of forward primer of target 3 (Rc-F3) attached to the 5′ terminus of forward primer of Target 1 (F1), together prime the amplification of Target 1. The resulting amplicon comprises the Target 1 nucleic acid sequence and also incorporates the forward primer sequence of target 3 (Rc-F3) into its 3′ terminus (the tag sequence). This amplicon in subsequent PCR cycles will prime the amplification of Target 3 as a forward primer for Target 3.

Similarly, the reverse primer of Target 2, i.e., the R2 primer and Intermediate Primer 2 (Rc-R3-F2) comprising the reverse complimentary sequence of reverse primer of Target 3 (Rc-R3) attached to the 5′ terminus of forward primer for Target 2 (F2), together prime the amplification of target 2. The resulting amplicon comprises Target 2 nucleic acid sequence and also incorporates the reverse primer sequence of target 3 into its 3′ terminus (tag sequence) and will prime the amplification from Target 3 as a reverse primer in subsequent PCR cycles.

As a result of the above PCR reactions with the four primers, i.e., primers R1, Rc-F3-F1, R2 and Rc-R3-F2, a fused product of amplicons comprising Targets 1, 2, and 3 will be produced. Detecting these fused amplicon products is an indication of the co-presence of Target 1, 2, and 3 in a sample that is tested using this method.

In some embodiments, a tag sequence can be either a forward or a reverse primer for the third target and can be attached to primers of either a first target or a second target. In some embodiments, a tag sequence can be attached to either a forward or a reverse primer of a first target and a second target.

In some embodiments, a method of the disclosure is designed to generate PCR primers for a third target from amplicons of two other targets (amplicons of target 1 and target 2 during initial PCR cycles as shown in FIG. 1). This creates an artificial gene that contains all three target genes. A single PCR assay/method is then able to detect the co-presence of all three targets using a forward primer from the first target and a reverse primer from the second target.

FIG. 2 shows an agarose gel electrophoresis showing the results of a method as depicted in FIG. 1 and shows the production of fused amplicons comprising three targets (Targets 1, 2 and 3) that were collectively detected to identify a Salmonella Hadar strain. Final concentrations of intermediate primers used affect the efficiency of the hopping PCR method. In the experiment performed to illustrate the method shown in FIG. 1, 1 μL of Salmonella Hadar strain DNA was used for 20 μL PCR reaction, using the TaqMan® Gene Expression Master Mix from Life Technologies (#4369016) and GeneAmp® PCR System 9700 from Life Technologies (#4.1445). The thermal profiles of the PCR reaction are: 95° C. 10 min; [95° C. 15 sec, 60° C. 45 sec]×10; [95° C. 15 sec, 60° C. 3 min]×10; and [95° C. 15 sec, 60° C. 45 sec]×40. Final concentrations of intermediate primers varied as shown on the top of the lanes in FIG. 2 from 0 nM to 300 nM. All other primers were used at 300 nM final concentrations. PCR products were run on 2% ethidium bromide agarose gel. As shown in FIG. 2. Fused amplicons comprising Target 1, 2 and 3 are detected in addition to Amplicons from Target 2 and Target 1.

FIGS. 3A and 3B illustrate real-time quantitative hopping PCR results that show in FIG. 3A high sensitivity and in FIG. 3B good linearity with initial input Salmonella Hadar DNA copy numbers. 1 μL of serially diluted Salmonella Hadar strain DNA were used for each 20 μL PCR mix, TaqMan® Gene Expression Master Mix from Life Technologies (#4369016) and AB7500 Real-Time PCR System from Life Technologies (#4351106) were used for PCR. The thermal profiles are: 95° C. 10 min; [95° C. 15 sec, 60° C. 45 sec]×10; [95° C. 15 sec, 60° C. 3 min]×10; and real-time detection cycles of [95° C. 15 sec, 60° C. 45 sec]×40. Final concentrations of intermediate primers are 30 nM, all other primers and probe are at 300 nM final concentrations.

In some embodiments, another version of a hopping PCR method was designed to amplify and detect the co-existence of two target nucleic acid sequences and is illustrated in FIG. 4, which is reproduced below for convenience

[chemical structure]

In the method of FIG. 4, three primers are used comprising: 1) an intermediate primer comprising a tag sequence of a reverse complimentary sequence of forward primer of Target 2 (Rc-F2) attached to the 5′ terminus of forward primer of target 1 (F1); 2) a reverse primer of Target 1 (R1); and reverse primer of target 2 (R2).

During the initial PCR cycles, reverse primer of target 1 (R1) primes the amplification from Target 1 and the resulting amplicon incorporates forward primer sequence of Target 2 (Rc-F2) in its 3′ terminus and will prime the amplification from Target 2 as a forward primer in subsequent PCR cycles. As a result, a fused product of amplicons comprising Target 1 and Target 2 will be produced. Detection of such a fused amplicon serves as an indicator of the co-presence of target 1 and 2 in a tested sample.

In some embodiments, a tag sequence in the example hopping PCR method of FIG. 4 can be either on a forward or a reverse primer for a second target, and paired with either a reverse or a forward primer of a second target correspondingly. In some embodiments, a tag sequence can be attached to either a forward or a reverse primer of a first target.

FIG. 5 depicts an agarose gel electrophoresis result of a method as illustrated in FIG. 4 and shows the production of fused amplicons of two targets (Targets 1 and 2) that when co-detected collectively indicate the presence of an O157:H7 E. coli strain organism. Lane 1 comprises the PCR reaction having standard forward and reverse primers for Target 1 and the reverse primer for Target 2 and produces only one amplicon from Target 1. Lane 2 has the PCR reaction comprising reverse primers of Target 1 and Target 2 and an intermediate primer that comprises the conjugation of the reverse complimentary sequence of forward primer of target 2 (as tag) and the forward primer sequence of target 1. As shown the PCR reaction of lane 2 produces a fused amplicon product comprising Targets 1 and 2. For this experiment 1 μL of O157:H7 E. coli strain DNA was used for a 20 μL PCR reaction using TaqMan® Gene Expression Master Mix from Life Technologies (#4369016) and GeneAmp® PCR System 9700 from Life Technologies (#4.1445). The thermal profiles used are: 95° C. 10 min; [95° C. 15 sec, 60° C. 45 sec]×10; [95° C. 15 sec, 60° C. 3 min-1])(10; and [95° C. 15 sec, 60° C. 45 sec]×40. Final concentration of the intermediate primer is 30 nM, all other primers are at 300 nM final concentrations. PCR products were run on 2% ethidium bromide agarose gel.

As illustrated in the example, in the method as illustrated in FIG. 4, a primer to a second target is generated from an amplicon of the first target. As a result, an artificial gene is generated that contains both the first and second target genes and a single PCR assay/method is able to detect the co-presence of two target genes using a primer from the first target and another primer from the second target.

In both the methods as illustrated in FIG. 1 and FIG. 4. endpoint agarose gel electrophoresis can reveal the PCR products comprising fused amplicons of the multiple target nucleic acid sequences to be detected and confirm the co-existence of the multiple target genes. By using dsuch as SYBR green or other double strand DNA intercalating dyes or by using a TaqMan® probe comprising a target nucleic acid sequence that is amplified during later cycles, the co-existence of multiple targets can also be quantified in real-time.

Table 1 below shows a summary of Inclusivity and Exclusivity evaluation of Salmonella Hadar using a hopping PCR method as described herein, also described in this example as a hopping TaqMan® assay. Within the tested samples, two S. Hadar strains were both tested positive with Ct of 16.5 and 18.6 respectively; while 188 non-S. Hadar Salmonella strains were all tested negative. In these experiments 1 μL of lysate of each strain was used for a 20 μL hopping PCR experiment using TaqMan® Gene Expression Master Mix from Life Technologies (#4369016) and AB7500 Real-Time PCR System from Life Technologies (#4351106). The thermal profiles are: 95° C. 10 min; [95° C. 15 sec, 60° C. 45 sec]×10; [95° C. 15 sec, 60° C. 3 min])×10; and real-time detection cycles of [95° C. 15 sec, 60° C. 45 sec]×40. Final concentrations of intermediate primers are 30 nM, all other primers and probe are at 300 nM final concentrations.

TABLE 1
Salmonella Hadar hopping PCR inclusivity
and Exclusivity assay results

| | Species | Sample # | result |
|---|---|---|---|
| 1 | Hadar | 2 | positive |
| 2 | S. paratyphi B | 27 | negative |
| 3 | S. typhimurium | 25 | negative |
| 4 | S. muenchen | 14 | negative |
| 5 | S. heidelberg | 13 | negative |
| 6 | S. saintpaul | 10 | negative |
| 7 | Typhimurium | 6 | negative |
| 8 | S. choleraesuis | 4 | negative |
| 9 | S. enteritidis | 4 | negative |
| 10 | Infantis | 3 | negative |
| 11 | S. derby | 3 | negative |
| 12 | S. dublin | 3 | negative |
| 13 | S. panama | 3 | negative |
| 14 | S. paratyphi C | 3 | negative |
| 15 | S. typhi | 3 | negative |
| 16 | Typhi | 3 | negative |
| 17 | Agona | 2 | negative |
| 18 | Montevideo | 2 | negative |
| 19 | Newport | 2 | negative |
| 20 | Panama | 2 | negative |
| 21 | S. arizonae | 2 | negative |
| 22 | S. bongori | 2 | negative |
| 23 | S. infantis | 2 | |
| 24 | S. miami | 2 | |
| 25 | S. montevideo | 2 | |
| 26 | S. newport | 2 | |
| 27 | S. pulloru | 2 | |
| 28 | S. stanley | 2 | |
| 29 | S. typhisuis | 2 | |
| 30 | S. wien | 2 | |
| 31 | Senftenberg | 2 | |
| 32 | 1.40:g.z51:- | 1 | |
| 33 | 11:b:e.n.x | 1 | |
| 34 | 16:z4.z32:- | 1 | |
| 35 | 38[k]:z35:- | 1 | |
| 36 | 40:z4.z24:- | 1 | |
| 37 | 42:f.g.t:- | 1 | |
| 38 | 45:a.e.n.x | 1 | |
| 39 | 45a,b:g.z51:- | 1 | |
| 40 | 501.2.3:k:z | 1 | |
| 41 | 58:d:z6 | 1 | |
| 42 | Agona | 1 | |
| 43 | Choleraesuis | 1 | |
| 44 | Derby | 1 | |
| 45 | Give | 1 | negative |
| 46 | Mbandaka | 1 | negative |
| 47 | Paratyphi C | 1 | negative |
| 48 | Poona | 1 | negative |
| 49 | Pullorum | 1 | negative |
| 50 | S. agona | 1 | negative |
| 51 | S. anatum | 1 | negative |
| 52 | S. brandenberg | 1 | negative |
| 53 | S. decatur(d) | 1 | negative |
| 54 | S. duisburg | 1 | negative |
| 55 | S. emek | 1 | negative |
| 56 | S. gallinarum | 1 | negative |
| 57 | S. haifa | 1 | negative |
| 58 | S. indiana | 1 | negative |
| 59 | S. paratyphi A | 1 | negative |
| 60 | S. reading | 1 | negative |
| 61 | S. rubislaw | 1 | negative |
| 62 | S. schwarzengrund | 1 | negative |
| 63 | S. sendai | 1 | negative |
| 64 | S. senftenberg | 1 | negative |
| 65 | S. thompson | 1 | negative |
| | Total 65 species. | 190 samples | |

The present disclosure describes novel compositions and methods for detection and in some embodiments for differential detection of microorganisms and strains, subtypes and serovars thereof as well as cells that are difficult to distinguish from each other by a one step assay and that require detection by detection of two or more unique nucleic acid sequences that are characteristic of that microorganism or cell to be detected.

In one embodiment, bioinformatic and direct DNA sequencing comparisons of several various microorganisms can be conducted to determine and identify two or more unique loci specific to different subtypes/serovars/strains (unique loci can be described herein as “serovar specific target nucleic acids” “subtype specific nucleic acids” or “strain specific nucleic acids”). Once multiple target nucleic acids are identified as constituting a signature of target nucleic acid sequence of a microorganism (or a cell), primers can be designed as described in the illustrative methods of FIG. 1 and FIG. 4 and a hopping PCR method of the present disclosure can be performed to detect the presence of multiple targets by a one step PCR method.

Some embodiments describe compositions comprising a set of primers or primer sets, e.g., three or four primers for use in an amplification process (e.g. PCR) for detection of multiple target sequences. Primer sets comprise at least a forward primer and at least a reverse primer that are operable to specifically hybridize to one target nucleic acid sequence. In some embodiments, primers described herein are suitable for simultaneous multiplex amplification reactions of two or more target nucleic acid sequences at the same time.

Sequences of these primers can be pre-determined by bioinformatic and/or DNA sequencing methods. Accordingly, the primer compositions described here are not limited by their DNA sequence and can be made for hybridization to a target nucleic acid sequence of any organism or cell in which one desired to detect multiple target nucleic acids simultaneously.

Some embodiments describe compositions comprising a set of PCR primers for detecting multiple target nucleic acid sequences comprising: a first PCR primer comprising a reverse primer of a first target nucleic acid sequence; a second PCR primer comprising a reverse primer of a second target nucleic acid sequence; and a third PCR primer comprising either the 5′ terminus of a forward primer of the first target nucleic acid sequence or the 5′ terminus of a forward primer of a second target nucleic acid sequence and a tag sequence attached thereto, the tag sequence comprising either a reverse complement of the forward primer of the second target nucleic acid sequence or comprising a reverse complement of the forward primer of the first target nucleic acid sequence. A set of PCR primers as described here can detect at least two targets nucleic acid sequences if present on a nucleic acid to be tested (e.g., a sample derived nucleic acid).

In one example embodiment, a set of primers operable to detect at least two target nucleic acid sequences if present on a nucleic acid to be tested can comprise: a first PCR primer comprising a reverse primer of a first target nucleic acid sequence; a second PCR primer comprising a reverse primer of a second target nucleic acid sequence; and a third PCR primer comprising the 5′ terminus of a forward primer of the first target nucleic acid sequence and the tag sequence attached thereto comprising a reverse complement of the forward primer of the second target nucleic acid sequence.

In another example embodiment, a set of primers operable to detect at least two target nucleic acid sequences if present on a nucleic acid to be tested can comprise: a first PCR primer comprising a reverse primer of a first target nucleic acid sequence; a second PCR primer comprising a reverse primer of a second target nucleic acid sequence; and a third PCR primer comprising the 5′ terminus of a forward primer of a second target nucleic acid sequence and the tag sequence comprising a reverse complement of the forward primer of the first target nucleic acid sequence.

A PCR primer composition of the disclosure, for detecting two targets, in some embodiments is described as comprising: a first PCR primer having a first contiguous nucleic acid sequence that can specifically hybridize to the 5′ terminus of a first target nucleic acid sequence joined to a second contiguous nucleic acid sequence that is a reverse complement of at least a part of the 5′ terminus of a second target nucleic acid sequence OR a first PCR primer having a first contiguous nucleic acid sequence that can specifically hybridize to the 5′ terminus of a first target nucleic acid sequence joined to a second contiguous nucleic acid sequence that is a reverse complement of at least a part of the 5′ terminus of a first target nucleic acid sequence; a second PCR primer that can specifically hybridize to the 3′ end of the first target nucleic acid sequence; a third PCR primer that is a reverse complement of at least a part of the 3′ terminus of the second target nucleic acid sequence.

Some embodiments describe compositions comprising a set of PCR primers for detecting multiple target nucleic acid sequences, such as more than two target nucleic acid sequences, comprising: a first PCR primer comprising a reverse primer of a first target nucleic acid sequence; a second PCR primer comprising a reverse primer of a second target nucleic acid sequence; a third PCR primer comprising the 5′ terminus of a forward primer of the first target nucleic acid sequence joined to a tag sequence comprising a reverse complement of the forward primer of a third target nucleic acid sequence or the third PCR primer comprising the 5′ terminus of a forward primer of the second target nucleic acid sequence joined to a tag sequence of the third PCR primer comprising a reverse complement of the forward primer of a third target nucleic acid sequence; and a fourth PCR primer comprising the 5′ terminus of a forward primer of the second target nucleic acid sequence joined to a tag sequence comprising a reverse complement of the reverse primer of the third target nucleic acid sequence or a fourth PCR primer comprising the 5′terminus of a forward primer of the first target nucleic acid sequence joined to a tag sequence of the fourth PCR primer comprising a reverse complement of the forward primer of a third target nucleic acid sequence.

In one example embodiment, a set of primers operable to detect at least three targets nucleic acid sequences if present on a nucleic acid to be tested can comprise: a first PCR primer comprising a reverse primer of a first target nucleic acid sequence; a second PCR primer comprising a reverse primer of a second target nucleic acid sequence; a third PCR primer comprising the 5′ terminus of a forward primer of the first target nucleic acid sequence joined to a tag sequence comprising a reverse complement of the forward primer of a third target nucleic acid sequence; and a fourth PCR primer comprising the 5′ terminus of a forward primer of the second target nucleic acid sequence joined to a tag sequence comprising a reverse complement of the reverse primer of the third target nucleic acid sequence.

In another example embodiment, a set of primers operable to detect at least three target nucleic acid sequences if present on a nucleic acid to be tested can comprise: a first PCR primer comprising a reverse primer of a first target nucleic acid sequence; a second PCR primer comprising a reverse primer of a second target nucleic acid sequence; a third PCR primer comprising the 5′ terminus of a forward primer of the second target nucleic acid sequence joined to a tag sequence of the third PCR primer comprising a reverse complement of the forward primer of a third target nucleic acid sequence; and a fourth PCR primer comprising the 5′ terminus of a forward primer of the first target nucleic acid sequence joined to a tag sequence of the fourth PCR primer comprising a reverse complement of the forward primer of a third target nucleic acid sequence.

In some embodiments, PCR primer compositions of the, to detect at least three target sequences, can also be described as a first PCR primer having a first contiguous nucleic acid sequence that can specifically hybridize to the 5′ terminus of a first target nucleic acid sequence joined to a second contiguous nucleic acid sequence that is a reverse complement of at least a part of the 5′ terminus of a third target nucleic acid sequence; a second PCR primer that can specifically hybridize to the 3′ end of the first target nucleic acid sequence; a third PCR primer having a first contiguous nucleic acid sequence that can specifically hybridize to the 5′ terminus of the second target nucleic acid sequence joined to a second contiguous nucleic acid sequence that is a reverse complement of at least a part of the 3′ terminus of a third target nucleic acid sequence; a fourth PCR primer that can specifically hybridize to the 3′ end of the second target nucleic acid sequence

Although illustrative examples have described detection of three and two targets, suitable modifications may be made to primer design in light of the present specification to detect four, five or more targets using the teachings of the present specification.

In some embodiments, primer compositions of the disclosure may further comprise one or more label, such as, but not limited to, a dye, a radioactive isotope, a chemiluminescent label, a fluorescent moiety, a bioluminescent label an enzyme, and combinations thereof.

In some embodiments, the present disclosure describes methods of detection of multiple nucleic acid sequences (multiple targets) in a sample nucleic acid comprising a single step nucleic acid amplification method such as a polymerase chain reaction (PCR) based methods. Such a method involves providing a plurality of primer compositions according the present disclosure specific to hybridize to at least two or more target nucleic acid sequences to be detected (in various embodiments, compositions comprising: at least two primers, or at least three primers, at least four primers, or more) and other components and conditions for a PCR reaction and generating one or more additional primers to prime one or more additional target nucleic acid sequences desired to be detected. Accordingly, some embodiments describe methods of generating a new PCR primer.

A method for generating a new PCR primer set, according to some embodiments, comprises: contacting a nucleic acid having three target nucleic acid sequences (e.g., a sample nucleic acid) with a plurality of primers comprising: a first PCR primer having a first contiguous nucleic acid sequence that can specifically hybridize to the 5′ terminus of a first target nucleic acid sequence joined to a second contiguous nucleic acid sequence that is a reverse complement of at least a part of the 5′ terminus of a third target nucleic acid sequence; a second PCR primer that can specifically hybridize to the 3′ end of the first target nucleic acid sequence; a third PCR primer having a first contiguous nucleic acid sequence that can specifically hybridize to the 5′ terminus of the second target nucleic acid sequence joined to a second contiguous nucleic acid sequence that is a reverse complement of at least a part of the 3′ terminus of a third target nucleic acid sequence; and a fourth PCR primer that can specifically hybridize to the 3′ end of the second target nucleic acid sequence; providing conditions for amplification of the first target nucleic acid sequence primed by the first and the second PCR primers to amplify and generate a first amplicon comprising the first target nucleic acid sequence and at least a part of the 5′ terminus of the third target nucleic acid sequence; and co-amplifying the second target nucleic acid sequence primed by the third and fourth PCR primers to amplify and generate a second amplicon comprising the second target nucleic acid sequence and at least a part of the 3′ end of the third target nucleic acid sequence, wherein the first and second amplicons generated comprise the new PCR primers (new PCR primer set) that can specifically hybridize to and prime the amplification of the 3′ and the 5′ ends of the third nucleic acid sequence in the same PCR reaction. Accordingly a method of the disclosure can generate in vivo a new primer set in initial PCR cycles (by using primer compositions of the disclosure). The new primer set generated then in later PCR cycles hybridizes to and amplifies a third target nucleic acid sequence to be detected. In some embodiments, detection of three target nucleic acids in a sample will comprise detecting one or more amplicons generated during the PCR reaction.

A method for generating a new PCR primer, set according to some embodiments, comprises: contacting a nucleic acid having two target nucleic acid sequences (e.g., a sample nucleic acid) with a plurality of primers comprising: a first PCR primer having a first contiguous nucleic acid sequence that can specifically hybridize to the 5′ terminus of a first target nucleic acid sequence joined to a second contiguous nucleic acid sequence that is a reverse complement of at least a part of the 5′ terminus of a second target nucleic acid sequence; a second PCR primer that can specifically hybridize to the 3′ end of the first target nucleic acid sequence; and a third PCR primer that is a reverse complement of at least a part of the 3′ terminus of the second target nucleic acid sequence; and providing conditions for amplification of the first target nucleic acid sequence primed by the first and the second PCR primers to amplify and generate an amplicon comprising the first target nucleic acid sequence and at least a part of the 5′ terminus of the second target nucleic acid sequence; wherein the amplicons generated comprises the new PCR primer that can specifically hybridize to and prime the amplification of the 5′ ends of the second target nucleic acid sequence and can be used with the third primer to amplify the second target nucleic acid. In some embodiments, a method of the disclosure as described here can generate in vivo a new primer in initial PCR cycles that in later PCR cycles can hybridizes to and amplify a second target nucleic acid sequence to be detected. In some embodiments, detection of two target nucleic acid sequences in a sample will comprise detecting one or more amplicons generated during the PCR reaction.

Some embodiments describe methods for detecting multiple target nucleic acids in a sample comprising: contacting sample derived nucleic acids with at least three PCR primers comprising: a first PCR primer having a first contiguous nucleic acid sequence that can specifically hybridize to the 5′ terminus of a first target nucleic acid sequence joined to a second contiguous nucleic acid sequence that is a reverse complement of at least a part of the 5′ terminus of a second target nucleic acid sequence; a second PCR primer that can specifically hybridize to the 3′ end of the first target nucleic acid sequence; and a third PCR primer that is a reverse complement of at least a part of the 3′ terminus of the second target nucleic acid sequence; amplifying the first target nucleic acid sequence primed by the first and the second PCR primers to generate an amplicon comprising the first target nucleic acid sequence and at least a part of the 5′ terminus of the second target nucleic acid sequence; amplifying the second target nucleic acid using the amplicon generated as a fourth primer with the third primer to hybridize to and prime the amplification of the second target nucleic acid sequence to generate a second amplicon comprising a fusion of the second target nucleic acid sequence and part of the first target nucleic acid sequence; and detecting the presence of the second amplicon to detect the presence of the first and the second nucleic acid sequences in the sample derived nucleic acids. In some embodiments, detection of two target nucleic acid sequences in a sample will comprise detecting one or more amplicons generated during the PCR reaction.

In some embodiments of the method, the first and the second target nucleic acid sequences comprise a signature of target nucleic acid sequences unique to a cell type such as a microorganism, a bacteria, a virus, a fungi, a pathogen, a subtype of a microorganism, a serotype of a microorganism or virus, a strain of a virus or microorganism, a diseased cell, a cancerous cell, a stem cell and can be used to specifically detect the unique cell type.

In some embodiments, methods for detecting multiple target nucleic acid sequences in a sample comprise: contacting a sample derived nucleic acid with at least four primers comprising: a first PCR primer having a first contiguous nucleic acid sequence that can specifically hybridize to the 5′ terminus of a first target nucleic acid sequence joined to a second contiguous nucleic acid sequence that is a reverse complement of at least a part of the 5′ terminus of a third target nucleic acid sequence; a second PCR primer that can specifically hybridize to the 3′ end of the first target nucleic acid sequence; a third PCR primer having a first contiguous nucleic acid sequence that can specifically hybridize to the 5′ terminus of the second target nucleic acid sequence joined to a second contiguous nucleic acid sequence that is a reverse complement of at least a part of the 3′ terminus of a third target nucleic acid sequence; and a fourth PCR primer that can specifically hybridize to the 3′ end of the second target nucleic acid sequence; amplifying the first target nucleic acid sequence primed by the first and the second PCR primers to amplify and generate a first amplicon comprising the first target nucleic acid sequence and at least a part of the 5′ terminus of the third target nucleic acid sequence; co-amplifying the second target nucleic acid sequence primed by the third and fourth PCR primers to amplify and generate a second amplicon comprising the second target nucleic acid sequence and at least a part of the 3′ end of the third target nucleic acid sequence, wherein the first and second amplicons generated comprise a new PCR primer set that can specifically hybridize to and prime the amplification of the third nucleic acid sequence; amplifying the third target nucleic acid using the first and second amplicons generated to generate a third amplicon comprising a fusion of the first, second and third target nucleic acid sequences; and detecting the presence of the third amplicon to detect the presence of the first, second and third nucleic acid sequences in the sample derived nucleic acids. In some embodiments, the first, second and the third target nucleic acid sequences comprise a signature of target nucleic acid sequences unique to a cell type such as a microorganism, a bacteria, a virus, a fungi, a pathogen, a subtype of a microorganism, a serotype of a microorganism or virus, a strain of a virus or microorganism, a diseased cell, a cancerous cell, a stem cell and can be used to specifically detect the unique cell type.

A method of the disclosure can further comprise processing a sample. In some embodiments this can comprise steps such as but not limited to: nucleic acid extraction; and/or nucleic acid purification from microorganisms in a sample; and/or optional enrichment of microorganisms in a sample prior to nucleic acid extraction and/or purification.

In some embodiment methods, a sample to be tested for potential contamination can be tested directly or can be “prepared” or “processed” in some manner prior to molecular testing and analysis (such as by PCR). For example, a sample can be processed to separate and/or to enrich a contaminating microbe. A sample can also be further processed to separate microbial nucleic acids from the remainder of the sample by lysing microbial cells. Lysing can be accomplished using a variety of buffers that can comprise lysing agents such as but not limited to chaotropic agents, and/or enzymatic agents and/or proteases. Lysed microbial cells from a sample can be additionally processed to separate, isolate and/or extract genetic material from the microbe prior to the amplification analysis methods described herein by several methods known to the skilled artisan. For example, nucleic acid extraction can be performed by kits and reagents from Life Technologies Corporation such as The PrepSEQ™ Rapid Spin Sample Preparation Kit can be used to prepare DNA from food and/or environmental samples for use in PCR amplification reactions. Using a simple spin protocol, the PrepSEQ™ Rapid Spin efficiently prepares microbial DNA from food matrices, by forming a lysate with the DNA (but not extracting DNA), that is compatible for PCR amplification. The kit provides a fast, cost-effective solution for preparing DNA from a broad range of sample types. The PrepSEQ® Nucleic Acid Extraction Kit from Life Technologies produces high-quality bacterial DNA samples for PCR-based detection from a wide range of food and environmental samples.

Some embodiments can comprise one or more of the following steps to achieve separation of microbes and/or their nucleic acids from sample components prior to analysis of microbial nucleic acids as described herein: (1) optional bacterial enrichment to enrich certain types of bacteria (e.g., by providing conditions to selectively increase one bacterial type), (2) optional bacterial cell lysis, (3) optional nucleic acid extraction and/or purification (e.g., DNA extraction, total nucleic acid extraction (i.e., DNA and RNA), genomic DNA extraction, RNA extraction using spin columns and/or buffers and/or other known methods in the art).

A method of the disclosure to co-detect the presence of two or more target nucleic acids by a single assay, in some embodiments, further comprises using a single detectable label for detection. For example, in the methods outlined above the amplicon comprising the fusion of target nucleic acid sequences, i.e., either a second amplicon comprising a fusion of the second target nucleic acid sequence and part of the first target nucleic acid sequence; or a third amplicon comprising a fusion of the first, second and third target nucleic acid sequences, can be detected by a single label. In some non-limiting examples, the single label can be a DNA intercalating dye such as ethedium bromide, or SYBR green or a TaqMan® dye.

In some embodiments, detection of amplification products can be done by hybridization of a labeled probe with specificity to an amplicon following an amplification reaction to detect the generation of an amplicon.

In some embodiments, a nucleic acid amplification can be preceded or simultaneously coupled with hybridization of a labeled probe, such as a TaqMan® probe, to a target nucleic acid prior to the commencement of the polymerase reaction.

Compositions and methods of the present disclosure are ideally suited for the preparation of kits.

One embodiment kit can comprising: a first PCR primer having a first contiguous nucleic acid sequence that can specifically hybridize to the 5′ terminus of a first target nucleic acid sequence joined to a second contiguous nucleic acid sequence that is a reverse complement of at least a part of the 5′ terminus of a second target nucleic acid sequence; a second PCR primer that can specifically hybridize to the 3′ end of the first target nucleic acid sequence; a third PCR primer that is a reverse complement of at least a part of the 3′ terminus of the second target nucleic acid sequence; and one or more components selected from a group consisting of: at least one enzyme, dNTPs, PCR reagents, at least one buffer, at least one salt, at least one control nucleic acid sample and an instruction protocol.

Another kit embodiment can comprise: a first PCR primer having a first contiguous nucleic acid sequence that can specifically hybridize to the 5′ terminus of a first target nucleic acid sequence joined to a second contiguous nucleic acid sequence (or tag sequence) that is a reverse complement of at least a part of the 5′ terminus of a third target nucleic acid sequence; a second PCR primer that can specifically hybridize to the 3′ end of the first target nucleic acid sequence; a third PCR primer having a first contiguous nucleic acid sequence that can specifically hybridize to the 5′ terminus of the second target nucleic acid sequence joined to a second contiguous nucleic acid sequence (or a tag sequence) that is a reverse complement of at least a part of the 3′ terminus of a third target nucleic acid sequence; a fourth PCR primer that can specifically hybridize to the 3′ end of the second target nucleic acid sequence; and one or more components selected from a group consisting of: at least one enzyme, dNTPs, PCR reagents, at least one buffer, at least one salt, at least one control nucleic acid sample and an instruction protocol.

In some embodiments, kit primers can be labeled. A kit comprising multiple pairs of primers can have primer pairs each labeled with different labels that can be detected separately. A kit can also comprise probes specific to detect one or more amplicons. Probes comprised in kits of the disclosure can be labeled. If a kit comprises multiple probes each probe can be labeled with a different label to allow detection of different amplification products that are specifically hybridized to by each different probe.

Kit components may be provided as solutions or as lyophilized powders which may be later reconstituted if needed in solutions and/or buffers which may also be provided. Components of kits may be individually and in various combinations comprised in one or a plurality of suitable container means. It is within the scope of these teachings to provide test kits for use in manual applications or test kits for use with automated sample preparation, reaction set-up, detectors or analyzers. In some embodiments, a kit amplification product may be further analyzed by methods such as but not limited to electrophoresis, hybridization, mass spectrometry, molecular barcoding, microfluidics, chemiluminescence and/or enzyme technologies.

Those having ordinary skill in the art, in light of this specification, will understand that many modifications, alternatives, and equivalents of the embodiments described above are possible. All such modifications, alternatives, and equivalents are intended to be encompassed herein.

All publications and patent applications cited above are incorporated by reference in their entirety for all purposes to the same extent as if each individual publication or patent application were specifically and individually indicated to be so incorporated by reference. Although the present disclosure has been described in some detail by way of illustration and example for purposes of clarity and understanding, it will be apparent that certain changes and modifications may be practiced within the scope of the appended claims.

What is claimed is:

1. A set of PCR primers for detecting multiple target nucleic acid sequences comprising:
a first PCR primer comprising a reverse primer of a first target nucleic acid sequence;
a second PCR primer comprising a reverse primer of a second target nucleic acid sequence; and
a third PCR primer comprising the 5′ terminus of a forward primer of the first target nucleic acid sequence or the 5′ terminus of a forward primer of a second target nucleic acid sequence and a tag sequence attached thereto, the tag sequence comprising a reverse complement of the forward primer of the second target nucleic acid sequence or comprising a reverse complement of the forward primer of the first target nucleic acid sequence.

2. The set of primers of claim 1 comprising:
a first PCR primer comprising a reverse primer of a first target nucleic acid sequence;
a second PCR primer comprising a reverse primer of a second target nucleic acid sequence; and
a third PCR primer comprising the 5′ terminus of a forward primer of the first target nucleic acid sequence and the tag sequence attached thereto comprising a reverse complement of the forward primer of the second target nucleic acid sequence.

3. The set of primers of claim 1 comprising:
a first PCR primer comprising a reverse primer of a first target nucleic acid sequence;
a second PCR primer comprising a reverse primer of a second target nucleic acid sequence; and
a third PCR primer comprising the 5′ terminus of a forward primer of a second target nucleic acid sequence and the tag sequence comprising a reverse complement of the forward primer of the first target nucleic acid sequence.

4. The set of PCR primers of claim 1 wherein the number of targets detected are two.

5. A set of PCR primers for detecting multiple target nucleic acid sequences comprising:
a first PCR primer comprising a reverse primer of a first target nucleic acid sequence;
a second PCR primer comprising a reverse primer of a second target nucleic acid sequence;
a third PCR primer comprising the 5′ terminus of a forward primer of the first target nucleic acid sequence joined to a tag sequence comprising a reverse complement of the forward primer of a third target nucleic acid sequence or the third PCR primer comprising the 5′ terminus of a forward primer of the second target nucleic acid sequence joined to a tag sequence of the third PCR primer comprising a reverse complement of the forward primer of a third target nucleic acid sequence; and
a fourth PCR primer comprising the 5′ terminus of a forward primer of the second target nucleic acid sequence joined to a tag sequence comprising a reverse complement of the reverse primer of the third target nucleic acid sequence or a fourth PCR primer comprising the 5′ terminus of a forward primer of the first target nucleic acid sequence joined to a tag sequence of the fourth PCR primer comprising a reverse complement of the forward primer of a third target nucleic acid sequence.

6. The set of primers of claim 5 comprising wherein the number of targets detected are three.

7. The set of PCR primers of claim 5 comprising:
a first PCR primer comprising a reverse primer of a first target nucleic acid sequence;
a second PCR primer comprising a reverse primer of a second target nucleic acid sequence;
a third PCR primer comprising the 5′ terminus of a forward primer of the first target nucleic acid sequence joined to a tag sequence comprising a reverse complement of the forward primer of a third target nucleic acid sequence; and
a fourth PCR primer comprising the 5′ terminus of a forward primer of the second target nucleic acid sequence joined to a tag sequence comprising a reverse complement of the reverse primer of the third target nucleic acid sequence.

8. The set of PCR primers of claim 5 comprising:
a first PCR primer comprising a reverse primer of a first target nucleic acid sequence;
a second PCR primer comprising a reverse primer of a second target nucleic acid sequence;
a third PCR primer comprising the 5′ terminus of a forward primer of the second target nucleic acid sequence joined to a tag sequence of the third PCR primer comprising a reverse complement of the forward primer of a third target nucleic acid sequence; and
a fourth PCR primer comprising the 5′ terminus of a forward primer of the first target nucleic acid sequence joined to a tag sequence of the fourth PCR primer comprising a reverse complement of the forward primer of a third target nucleic acid sequence.

9. A method for generating a new PCR primer set comprising:
contacting a nucleic acid having three target nucleic acid sequences with a plurality of primers comprising:
a first PCR primer having a first contiguous nucleic acid sequence that can specifically hybridize to the 5′ terminus of a first target nucleic acid sequence joined to a second contiguous nucleic acid sequence that is a reverse complement of at least a part of the 5′ terminus of a third target nucleic acid sequence;
a second PCR primer that can specifically hybridize to the 3′ end of the first target nucleic acid sequence;
a third PCR primer having a first contiguous nucleic acid sequence that can specifically hybridize to the 5′ terminus of the second target nucleic acid sequence joined to a second contiguous nucleic acid sequence that is a reverse complement of at least a part of the 3′ terminus of a third target nucleic acid sequence; and
a fourth PCR primer that can specifically hybridize to the 3′ end of the second target nucleic acid sequence;
providing conditions for amplification of the first target nucleic acid sequence primed by the first and the second PCR primers to amplify and generate a first amplicon comprising the first target nucleic acid sequence and at least a part of the 5′ terminus of the third target nucleic acid sequence; and
co-amplifying the second target nucleic acid sequence primed by the third and fourth PCR primers to amplify and generate a second amplicon comprising the second target nucleic acid sequence and at least a part of the 3′ end of the third target nucleic acid sequence,
wherein the first and second amplicons generated comprise the new PCR primer set that can specifically hybridize to and prime the amplification of the 3′ and the 5′ ends of the third nucleic acid sequence.

10. A method for generating a new PCR primer comprising:
contacting a nucleic acid having two target nucleic acid sequences with a plurality of primers comprising:
a first PCR primer having a first contiguous nucleic acid sequence that can specifically hybridize to the 5′ terminus of a first target nucleic acid sequence joined to a second contiguous nucleic acid sequence that is a reverse complement of at least a part of the 5′ terminus of a second target nucleic acid sequence;
a second PCR primer that can specifically hybridize to the 3′ end of the first target nucleic acid sequence; and
a third PCR primer that is a reverse complement of at least a part of the 3′ terminus of the second target nucleic acid sequence; and
providing conditions for amplification of the first target nucleic acid sequence primed by the first and the second PCR primers to amplify and generate an amplicon comprising the first target nucleic acid sequence and at least a part of the 5′ terminus of the second target nucleic acid sequence;
wherein the amplicons generated comprises the new PCR primer that can specifically hybridize to and prime the amplification of the 5′ ends of the second target nucleic acid sequence and can be used with the third primer to amplify the second target nucleic acid.

11. A method for detecting multiple target nucleic acids in a sample comprising:
contacting sample derived nucleic acids with at least three PCR primers comprising:
a first PCR primer having a first contiguous nucleic acid sequence that can specifically hybridize to the 5′ terminus of a first target nucleic acid sequence joined to a second contiguous nucleic acid sequence that is a reverse complement of at least a part of the 5′ terminus of a second target nucleic acid sequence;
a second PCR primer that can specifically hybridize to the 3′ end of the first target nucleic acid sequence; and
a third PCR primer that is a reverse complement of at least a part of the 3′ terminus of the second target nucleic acid sequence;
amplifying the first target nucleic acid sequence primed by the first and the second PCR primers to generate an amplicon comprising the first target nucleic acid sequence and at least a part of the 5′ terminus of the second target nucleic acid sequence;
amplifying the second target nucleic acid using the amplicon generated as a fourth primer with the third primer to hybridize to and prime the amplification of the second target nucleic acid sequence to generate a second amplicon comprising a fusion of the second target nucleic acid sequence and part of the first target nucleic acid sequence; and
detecting the presence of the second amplicon to detect the presence of the first and the second nucleic acid sequences in the sample derived nucleic acids.

12. The method of claim 11, wherein the first and the second target nucleic acid sequences comprise a signature of target nucleic acid sequences unique to a cell type such as a microorganism, a bacteria, a virus, a fungi, a pathogen, a subtype of a microorganism, a serotype of a microorganism or virus, a strain of a virus or microorganism, a diseased cell, a cancerous cell, a stem cell and can be used to specifically detect the unique cell type.

13. The method of claim 11, wherein the cell is a Salmonella enterica serovar.

14. The method of claim 11, wherein the cell is a S. Hadar.

15. The method of claim 11, wherein the cell is an E coli O157:H7.

16. A method for detecting multiple target nucleic acid sequences in a sample comprising:
contacting a sample derived nucleic acid with at least four primers comprising:
a first PCR primer having a first contiguous nucleic acid sequence that can specifically hybridize to the 5′ terminus of a first target nucleic acid sequence joined to a second contiguous nucleic acid sequence that is a reverse complement of at least a part of the 5′ terminus of a third target nucleic acid sequence;
a second PCR primer that can specifically hybridize to the 3′ end of the first target nucleic acid sequence;
a third PCR primer having a first contiguous nucleic acid sequence that can specifically hybridize to the 5′ terminus of the second target nucleic acid sequence joined to a second contiguous nucleic acid sequence that is a reverse complement of at least a part of the 3′ terminus of a third target nucleic acid sequence; and
a fourth PCR primer that can specifically hybridize to the 3′ end of the second target nucleic acid sequence;
amplifying the first target nucleic acid sequence primed by the first and the second PCR primers to amplify and generate a first amplicon comprising the first target nucleic acid sequence and at least a part of the 5′ terminus of the third target nucleic acid sequence;
co-amplifying the second target nucleic acid sequence primed by the third and fourth PCR primers to amplify and generate a second amplicon comprising the second target nucleic acid sequence and at least a part of the 3′ end of the third target nucleic acid sequence, wherein the first and second amplicons generated comprise a new PCR primer set that can specifically hybridize to and prime the amplification of the third nucleic acid sequence;
amplifying the third target nucleic acid using the first and second amplicons generated to generate a third amplicon comprising a fusion of the first, second and third target nucleic acid sequences; and
detecting the presence of the third amplicon to detect the presence of the first, second and third nucleic acid sequences in the sample derived nucleic acids.

17. A method of claim 13, wherein the first, second and the third target nucleic acid sequences comprise a signature of target nucleic acid sequences unique to a cell type such as a microorganism, a bacteria, a virus, a fungi, a pathogen, a subtype of a microorganism, a serotype of a microorganism or virus, a strain of a virus or microorganism, a diseased cell, a cancerous cell, a stem cell and can be used to specifically detect the unique cell type.

18. The method of claim 11, wherein the cell is a Salmonella enterica serovar.

19. The method of claim 11, wherein the cell is a S. Hadar.

20. The method of claim 11, wherein the cell is an E coli O157:H7.

21. A kit for detection of multiple target nucleic acid sequences in a sample derived nucleic acid comprising:
a first PCR primer having a first contiguous nucleic acid sequence that can specifically hybridize to the 5′ terminus of a first target nucleic acid sequence joined to a second contiguous nucleic acid sequence that is a reverse complement of at least a part of the 5′ terminus of a second target nucleic acid sequence;
a second PCR primer that can specifically hybridize to the 3′ end of the first target nucleic acid sequence;
a third PCR primer that is a reverse complement of at least a part of the 3′ terminus of the second target nucleic acid sequence; and
one or more components selected from a group consisting of: at least one enzyme, dNTPs, PCR reagents, at least one buffer, at least one salt, at least one control nucleic acid sample and an instruction protocol.

22. A kit for detection of multiple target nucleic acid sequences in a sample derived nucleic acid comprising:
a first PCR primer having a first contiguous nucleic acid sequence that can specifically hybridize to the 5′ terminus of a first target nucleic acid sequence joined to a second contiguous nucleic acid sequence that is a reverse complement of at least a part of the 5′ terminus of a third target nucleic acid sequence;
a second PCR primer that can specifically hybridize to the 3′ end of the first target nucleic acid sequence;
a third PCR primer having a first contiguous nucleic acid sequence that can specifically hybridize to the 5′ terminus of the second target nucleic acid sequence joined to a second contiguous nucleic acid sequence that is a reverse complement of at least a part of the 3′ terminus of a third target nucleic acid sequence;
a fourth PCR primer that can specifically hybridize to the 3′ end of the second target nucleic acid sequence; and
one or more components selected from a group consisting of: at least one enzyme, dNTPs, PCR reagents, at least one buffer, at least one salt, at least one control nucleic acid sample and an instruction protocol.
